(19) United States
(12) Patent Application Publication
(10) Pub. No.: US 2019/0076063 A1
(43) Pub. Date: Mar. 14, 2019

(54) SYSTEMS AND METHODS OF SKI ACTIVITY DETECTION

(71) Applicant: Apple Inc., Cupertino, CA (US)

(72) Inventors: Ryan P. Kent, San Mateo, CA (US); Karthik Jayaraman Raghuram, Mountain View, CA (US); Stephen P. Jackson, San Francisco, CA (US); Umamahesh Srinivas, Milpitas, CA (US); Ahmad Bleik, San Jose, CA (US); Bharath Narasimha Rao, Mountain View, CA (US)

(73) Assignee: Apple Inc., Cupertino, CA (US)

(21) Appl. No.: 16/106,763

(22) Filed: Aug. 21, 2018

Related U.S. Application Data
(60) Provisional application No. 62/556,782, filed on Sep. 11, 2017.

(51) Int. Cl. A61B 5/11; A61B 5/024; A63B 24/00

(52) U.S. Cl. CPC A61B 5/1118; A61B 5/02416; A63B 2024/0065; A61B 5/1112; A61B 5/02438; A63B 24/0062

(57) ABSTRACT

The present disclosure relates to a system and method for improving an accuracy of a wearable device while detecting a ski activity by a user at a ski area. In one aspect, the method can include receiving motion data of the user from a motion sensing module of the wearable device. A heart rate sensing module can measure a heart rate of the user. One or more processor circuits can detect the user is gripping ski poles on a substantially flat ground based on the motion data and the heart rate of the user. The one or more processor circuits can calculate information about the user's performance during the ski activity and output the information about the user's performance during the ski activity.

### CROSS-REFERENCE TO RELATED APPLICATIONS

This application claims priority to U.S. Provisional Application Ser. No. 62/556,782 filed Sep. 11, 2017, which is hereby incorporated by reference in its entirety.

### FIELD

The present disclosure relates generally to detecting ski activity using a wearable device.

### BACKGROUND

A wearable device may be worn on the hand, wrist, or arm of a person when skiing. It may be desirable to track ski activity by a user to promote exercise and for other health-related reasons.

### SUMMARY

The present disclosure describes systems and methods of detecting ski activity using wearable device. During a typical ski day, a skier may spend many hours (e.g., 8 hours) at a ski area, while actively skiing for only a fraction of that time. Between ski runs, the skier may spend a significant amount of time riding lifts, taking breaks, or otherwise being inactive. Due to for example, size and cost constraints, cost, the battery capacity of a wearable device may be such that it is not possible to run the device at full power for a full ski day without having to recharge the device's battery. Certain components of the wearable device—such as the main processor, Global Positioning System (GPS) receiver, and cellular module—can draw a particularly high amount of power. Accordingly, the systems and methods disclosed herein can detect when the user is riding a lift, taking a lunch break, or otherwise being inactive, and, in response, selectively power down certain components of the wearable device. When the user resumes skiing, the systems and methods can automatically return power (or “wake up”) to these components so as to accurately track the user's ski activity. The disclosure includes techniques for preventing waking up components too soon (i.e., preventing “false positives”), further conserving battery charge. Using the systems, methods, and techniques disclosed herein, a wearable device may track ski activity over a full ski day.

Other features and advantages will become apparent from the following detailed description and drawings.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1 is a diagram of an illustrative wearable device, in accordance with an embodiment of the present disclosure.

FIG. 2 is a block diagram showing example components that may be found within a wearable device, in accordance with an embodiment of the present disclosure.

FIG. 3 is a diagram of an illustrative companion device, in accordance with an embodiment of the present disclosure.

FIG. 4 is a flow chart illustrating a method for calculating performance information for a skier, in accordance with an embodiment of the present disclosure.

FIGS. 5 and 6 are flow charts illustrating methods for detecting a start of ski activity, in accordance with embodiments of the present disclosure.

FIGS. 7-10 are graphs showing sample motion data associated with ski activity, in accordance with embodiments of the present disclosure.

FIG. 11 is a flow chart illustrating a method for detecting ski lift/run activity, in accordance with an embodiment of the present disclosure.

FIGS. 12-14 are diagrams of state machines that may be used to detect ski lift/run activity, in accordance with embodiments of the present disclosure.

FIG. 15 is a flow chart illustrating a method for detecting a start/end of a lift, in accordance with an embodiment of the present disclosure.

FIG. 16 is a flow chart illustrating a method for crowdsourcing lift information, in accordance with an embodiment of the present disclosure.

FIG. 17 is a flow chart illustrating a method for determining a skier's ability, in accordance with an embodiment of the present disclosure.

FIG. 18 is a flow chart illustrating a method for crowdsourcing lift information, in accordance with an embodiment of the present disclosure.

FIG. 19 is a flow chart illustrating a method for providing a lift recommendation to a skier, in accordance with an embodiment of the present disclosure.

FIG. 20 is a flow chart illustrating a method for providing turn-by-turn navigation to a skier, in accordance with an embodiment of the present disclosure.

### DETAILED DESCRIPTION

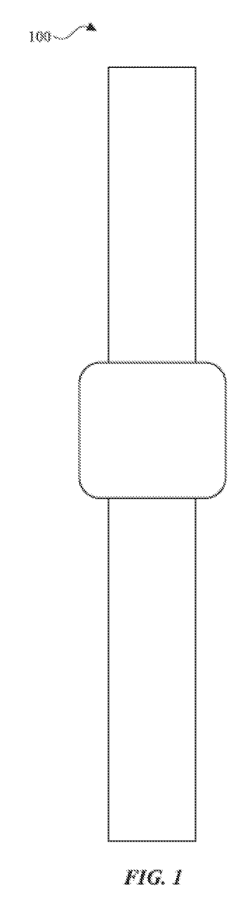

FIG. 1 shows an example of a wearable device 100 that may be worn by a skier (or “user”), in accordance with an embodiment of the present disclosure. In some embodiments, the wearable device 100 may be configured to be worn around the user's wrist using a strap (e.g., a watch strap).

As described in more detail below, the wearable device 100 may be configured to detect the user's ski activity, calculate performance information of the user while skiing, and provide additional ski-related functionality to the user. In particular, the wearable device 100 may use motion data obtained from motion sensors to detect when the user begins skiing, approaches a ski lift, gets on/off a lift, starts/stops a downhill ski run, makes turns, falls while skiing, among other ski-related activity. The wearable device may use a variety of motion data, including, in some embodiments, motion data from a companion device.

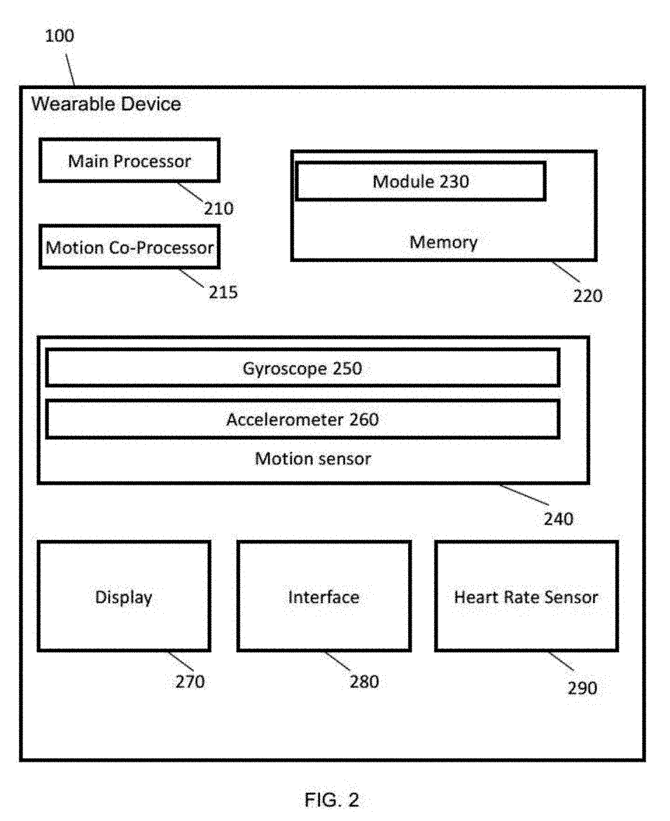

FIG. 2 depicts a block diagram of exemplary components that may be found within the wearable device 100 according to some embodiments of the present disclosure. In some embodiments, the wearable device 100 can include a main processor 210 (or “application processor” or “AP”), a motion co-processor 215, a memory 220, one or more motion sensors 240, a display 270, an interface 280, and a heart rate sensor 290. Wearable device 100 may include additional modules, fewer modules, or any other suitable combination of modules that perform any suitable operation or combination of operations.

In some embodiments, main processor 210 can include one or more cores and can accommodate one or more threads to run various applications and modules. Software can run on main processor 210 capable of executing computer instructions or computer code. Main processor 210 can also be implemented in hardware using an application specific integrated circuit (ASIC), programmable logic array (PLA), field programmable gate array (FPGA), or any other integrated circuit.

In some embodiments, wearable device 100 can also include a motion co-processor 215 which may draw less power than the main processor 210. Whereas the main processor 210 may be configured for general purpose computations and communications, the motion co-processor 215 may be configured to perform a relatively limited set of tasks, such as receiving and processing data from motion sensor 240, heart rate sensor 290, and other modules within the wearable device 100. In many embodiments, the main processor 210 may be powered down at certain times to conserve battery charge, while the motion co-processor 215 remains powered on. Thus, the motion co-processor 215 is sometimes referred to as an “always-on” processor (AOP). Motion co-processor 215 may control when the main processor 210 is powered on or off.

Memory 220 can be a non-transitory computer readable medium, flash memory, a magnetic disk drive, an optical drive, a programmable read-only memory (PROM), a read-only memory (ROM), or any other memory or combination of memories. Memory 220 can include one or more modules 230.

Main processor 210 or motion co-processor 215 can be configured to run module 230 stored in memory 220 that is configured to cause main processor 210 or motion co-processor 215 to perform various steps that are discussed throughout the present disclosure.

In some embodiments, wearable device 100 can include one or more motion sensors 240. For example, motion sensors 240 can include a gyroscope 250 and an accelerometer 260. In some embodiments, accelerometer 260 may be a three-axis accelerometer that measures linear acceleration in up to three-dimensions (for example, x-axis, y-axis, and z-axis). In some embodiments, gyroscope 250 may be a three-axis gyroscope that measures rotational data, such as rotational movement and/or angular velocity, in up to three-dimension (for example, yaw, pitch, and roll). In some embodiments, accelerometer 260 may be a microelectromechanical system (MEMS) accelerometer, and gyroscope 250 may be an MEMS gyroscope. Main processor 210 or motion co-processor 215 of wearable device 100 may receive motion information from one or more motion sensors 240 to track acceleration, rotation, position, or orientation information of wearable device 100 in six degrees of freedom through three-dimensional space.

In some embodiments, wearable device 100 may include other types of sensors in addition to accelerometer 260 and gyroscope 250. For example, wearable device 100 may include an altimeter or barometer, or other types of location sensors, such as a Global Positioning System (GPS) sensor.

Wearable device 100 may also include display 270. Display 270 may be a screen, such as a crystalline (e.g., sapphire) or glass touchscreen, configured to provide output to the user as well as receive input from the user via touch. For example, display 270 may be configured to display a current heart rate or daily average energy expenditure. Display 270 may receive input from the user to select, for example, which information should be displayed, or whether the user is beginning a physical activity (e.g., starting a session) or ending a physical activity (e.g., ending a session), such as a swimming session, a running session, or a skiing session. In some embodiments, wearable device 100 may present output to the user in other ways, such as by producing sound with a speaker (not shown), and wearable device 100 may receive input from the user in other ways, such as by receiving voice commands via a microphone (not shown).

In some embodiments, wearable device 100 may communicate with external devices via interface 280, including a configuration to present output to a user or receive input from a user. Interface 280 may be a wireless interface. The wireless interface may be a standard Bluetooth® (IEEE 802.15) interface, such as Bluetooth® v4.0, also known as “Bluetooth low energy.” In other embodiments, the interface may operate according to a cellphone network protocol such as Long Term Evolution (LTE™) or a Wi-Fi (IEEE 802.11) protocol. In other embodiments, interface 280 may include wired interfaces, such as a headphone jack or bus connector (e.g., Lightning®, Thunderbolt™, USB, etc.).

Wearable device 100 can measure an individual's current heart rate from heart rate sensor 290. Heart rate sensor 290 may also be configured to determine a confidence level indicating a relative likelihood of an accuracy of a given heart rate measurement. In other embodiments, a traditional heart rate monitor may be used and may communicate with wearable device 100 through a near field communication method (e.g., Bluetooth).

In some embodiments, the wearable device can include a photoplethysmogram (PPG) sensor. PPG is a technique for measuring a person's heart rate by optically measuring changes in the person's blood flow at a specific location. PPG can be implemented in many different types of devices in various forms and shapes. For example, a PPG sensor can be implemented in a wearable device in the form of a wrist strap, which a user can wear around the wrist. The PPG sensor can optically measure the blood flow at the wrist. Based on the blood flow information, the wrist strap or another connected device can derive the person's heart rate.

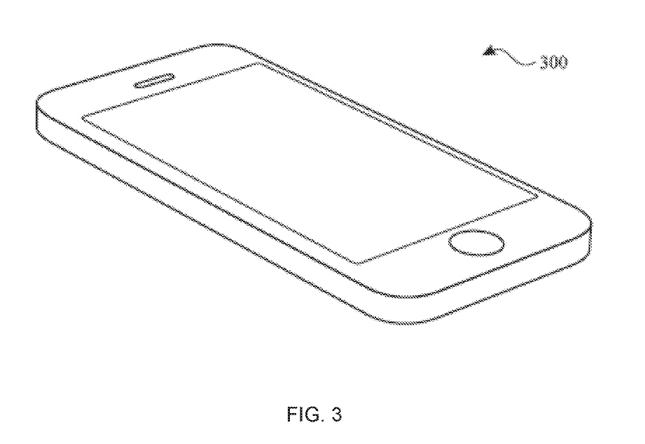

Wearable device 100 may be configured to communicate with a companion device 300 (FIG. 3), such as a smartphone, as described in more detail herein. In some embodiments, wearable device 100 may be configured to communicate with other external devices, such as a notebook or desktop computer, tablet, headphones, Bluetooth headset, etc.

The modules described above are examples, and embodiments of wearable device 100 may include other modules not shown. For example, some embodiments of wearable device 100 may include a rechargeable battery (e.g., a lithium-ion battery), a microphone or a microphone array, one or more cameras, one or more speakers, a watchband, water-resistant casing or coating, etc. In some embodiments, all modules within wearable device 100 can be electrically and/or mechanically coupled together. In some embodiments, main processor 210 can coordinate the communication among each module.

FIG. 3 shows an example of a companion device 300 according to some embodiments of the present disclosure. Wearable device 100 may be configured to communicate with companion device 300 via a wired or wireless communication channel (e.g., Bluetooth, Wi-Fi, etc.) In some embodiments, companion device 300 may be a smartphone, tablet computer, or similar portable computing device. Companion device 300 may be stored in the user's jacket/pants pocket, strapped to the user's arm with an armband or similar device, or otherwise positioned on the user's body while skiing and in communicable range of wearable device 100.

In some embodiments, companion device 300 may include a variety of sensors, such as location and motion sensors (not shown). When companion device 300 is available for communication with wearable device 100, wearable device 100 may receive additional data from companion device 300 to improve or supplement its calibration or calorimetry processes. For example, in some embodiments, wearable device 100 may not include a GPS sensor as opposed to an alternative embodiment in which wearable device 100 may include a GPS sensor. In the case where wearable device 100 may not include a GPS sensor, a GPS sensor of companion device 300 may collect GPS location information, and wearable device 100 may receive the GPS location information via interface 280 (FIG. 2) from companion device 300.

In another example, wearable device 100 may not include an altimeter or barometer, as opposed to an alternative embodiment in which wearable device 100 may include an altimeter or barometer. In the case where wearable device 100 may not include an altimeter or barometer, an altimeter or barometer of companion device 300 may collect altitude or relative altitude information, and wearable device 100 may receive the altitude or relative altitude information via interface 280 (FIG. 2) from the companion device 300.

In another example, wearable device 100 may receive motion information from companion device 300. Wearable device 100 may compare the motion information from companion device 300 with motion information from one or more motion sensors 240 of wearable device 100. Motion information such as data from accelerometer 260 and/or gyroscope 250 may be filtered (e.g. by a high-pass, low-pass, band-pass, or band-stop filter) in order to improve the quality of motion information. For example, a low-pass filter may be used to remove some ambient noise.

Wearable device 100 may use sensed and collected motion information to predict a user's activity. Examples of activities may include, but are not limited to, walking, running, cycling, swimming, skiing, etc. Wearable device 100 may also be able to predict or otherwise detect when a user is sedentary (e.g., sleeping, sitting, standing still, driving or otherwise controlling a vehicle, etc.) Wearable device 100 may use a variety of motion information, including, in some embodiments, motion information from a companion device.

Wearable device 100 may use a variety of heuristics, algorithms, or other techniques to predict the user's activity. Wearable device 100 may also estimate a confidence level (e.g., percentage likelihood, degree of accuracy, etc.) associated with a particular prediction (e.g., 90% likelihood that the user is skiing) or predictions (e.g., 60% likelihood that the user is skiing and 40% likelihood that the user is performing some other activity).

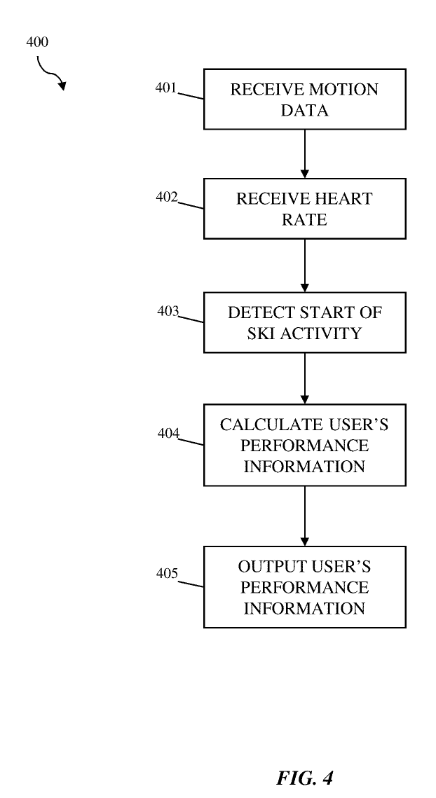

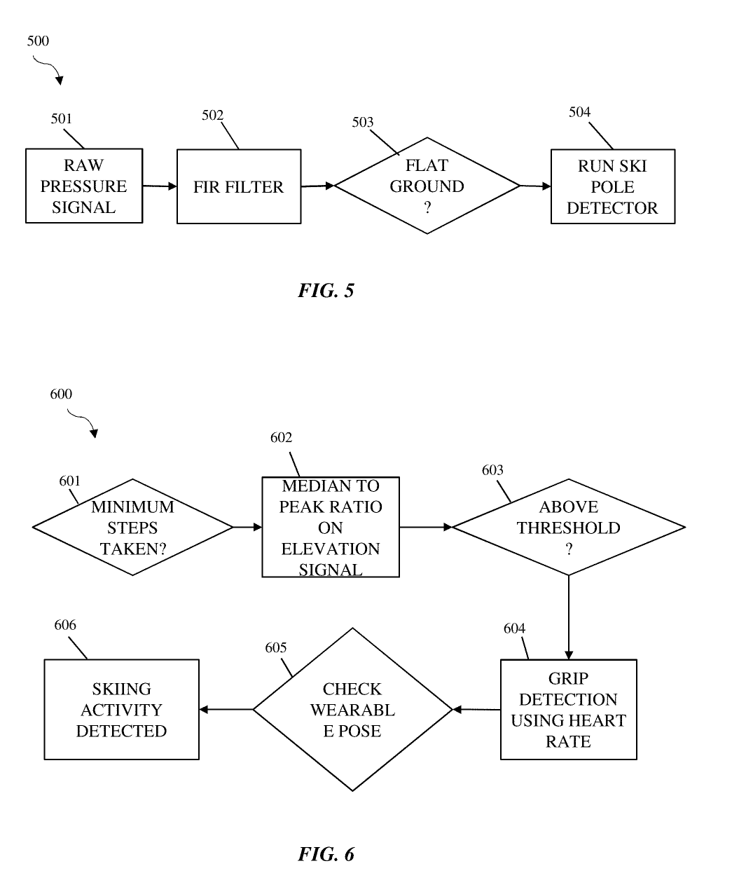

FIG. 4 shows a method 400 for calculating performance information for a skier, according to some embodiments of the present disclosure. At step 401, motion data of the user is received, for example, from motion sensor 240 of wearable device 100 (FIG. 2). The motion data may include accelerometer or gyroscope data according to some embodiments. At step 402, heart rate data of the user is received, for example, from heart rate sensor 290 of wearable device 100 (FIG. 2). At step 403, a start of a ski activity is detected using the motion and heart rate data. In many embodiments, method 400 determines the start of ski activity by detecting the user has gripped and/or planted ski poles, such as described below with respect to FIGS. 5 and 6. In some embodiments, heart rate data and motion data need can be acquired and processed in parallel.

Once the user has started skiing, various ski-related performance information of the user may be calculated at step 404. Non-limiting examples of performance information include a number of runs taken, a number of turns performed, descent rate, time spent on ski runs, time spent on ski lifts, and skiing ability. At step 405, ski performance information is output to the user, such as via the wearable device's display module 270 (FIG. 2). In some embodiments, the wearable device may provide additional functionality to the skier after detecting a start of ski activity. For example, the device may recommend lifts/runs for the user to take based on the user's skiing ability or may provide turn-by-run directions for the skier to navigate a ski area.

FIGS. 5 and 6 show methods that can be used together to detect a start of ski activity, according to some embodiments of the present disclosure. The methods are based on detecting, using motion data, determining that a user has gripped and/or planted ski poles in order to differentiate ski activity from snowboarding or other downhill activity. Various heuristics can be used to detect ski pole usage, such as the user's arm pose angle, the user's grip, pedestrian motion (i.e., “steps”), and/or spikes in atmospheric pressure data resulted from “jacket billowing.”

FIG. 5 shows a method 500 for ski pole detection, according to some embodiments of the present disclosure. At step 501, atmospheric pressure data (also referred to herein as “elevation data” or a “raw pressure signal”) is obtained from a pressure sensor (e.g., a barometer). At step 502, the raw pressure signal can be filtered to improve data quality. In particular, raw pressure data can be noisy, so a filter is used to smooth the signal in order to get a more accurate estimate of elevation change. In some embodiments, a finite impulse response (FIR) filter can be used. At step 503, a determination is made as to whether the user is on flat ground based on the filtered pressure signals. If the user is on flat ground, then a ski pole detector may be run at step 504. According to some embodiments, the ski pole detector of FIG. 6 may be used. If the user is not on flat ground, then the method 500 may terminate without determining a start of ski activity. This decision process is based on intuition that a skier will most likely be using poles if on flat ground.

FIG. 6 shows a method 600 for ski pole detection, according to some embodiments of the present disclosure. At step 601, a determination is made as to whether the user has taken at least minimum number of “steps” within a certain time period. In some embodiments, a pedometer within the wearable/companion device can be used to count the user's steps. Because a pole plant may involve similar arm movement as a pedestrian step, step count can be used as a proxy for a pole plants. In some embodiments, the minimum step count is two (2). In some embodiments, the pre-defined time period can be between two and three seconds (e.g., 2.56 seconds).

At step 602, a median-to-peak elevation ratio may be calculated based on a filtered pressure signal (e.g., the pressure signal from block 502 in FIG. 5). At step 603, the computed median-to-peak elevation ratio can be compared with a threshold value (e.g., a threshold value in the range of 5-7 feet). The median to peak elevation ratio can indicate whether the user is at the top of a ski run and ready to ski. If the computed ratio exceeds the threshold value, the ski detection method can proceed to step 604.

At step 604, heart rate data is used to detect whether the user is gripping ski poles. In some embodiments, the heart rate sensor can be a PPG sensor and it can detecting grip based on the “white knuckle” effect. If it is determined that the user is gripping poles, the ski detection method can proceed to step 605. “White knuckle” describes vascular occlusion due to active muscle groups. The “white knuckle” effect can typically occur when an individual grips tightly on an object, such as a dumbbell, bicycle handle, ski pole, etc. One area where this effect can be easily observed is around the knuckles (interphalangeal and metacarpophalangeal joints) when performing activities requiring gripping. The “white knuckle” effect can affect the accuracy of PPG-measured heart rate. In particular, the “white knuckle” effect can create noise artifacts that can interfere with heart rate detection by PPG because these artifacts can limit the ability to detect the influx and outflow of blood among the various tissues being analyzed. In some embodiments, monitoring these artifacts can allow the heart rate sensor to detect a user's pole gripping.

In some embodiments, detecting whether the user is gripping ski poles includes using techniques described in U.S. patent application Ser. No. 15/692,736, filed on Aug. 31, 2017, and entitled “SYSTEMS AND METHODS FOR DETERMINING AN INTENSITY LEVEL OF AN EXERCISE USING PHOTOPLETHYSMOGRAM (PPG),” which patent application is incorporated herein in its entirety.

At step 605, the user's wrist/arm pose can be determined. In some embodiments, accelerometer 260 of wearable device 100 (FIG. 2) can be used to determine the user's arm pose. For example, referring to FIG. 1, a body-fixed frame of reference can be defined with respect to wearable device 100. The z-axis can be perpendicular to the display surface of wearable device 100. The x-axis can be chosen to align with the direction which the crown is pointing at. The y-axis can be chosen to be perpendicular to both x and z axes. The wearable device 100 can detect gravity direction along the y-axis in the body-fixed frame of reference to determine the user's arm pose.

At step 606, a determination is made as to whether the user is skiing based on all the results from previous steps (i.e., pedometer, elevation, grip, and pose).

In some embodiments, a skier's arm swings when using poles can create spikes in filtered pressure signal, which can be attributed to “jacket billowing” effect when the cuff of the user's ski jacket is sealed around the watch. The pressure spikes can help to identify ski pole use.

It is appreciated that a user may use poles during other outdoor activities, not just skiing. For example, hikers commonly use poles when descending a mountain.

In order to differentiate skiing from such activities, various other factors may be considered. In some embodiments, location data from the wearable/companion device may be used to determine if the user proximate to a known ski area. In certain embodiments, season information can also be used to determine if the user is skiing or hiking. In particular embodiments, the user's ascent/descent rate, which can be calculated from changes in pressure data, can be used to determine if the user is skiing versus performing some other mountain activity with poles.

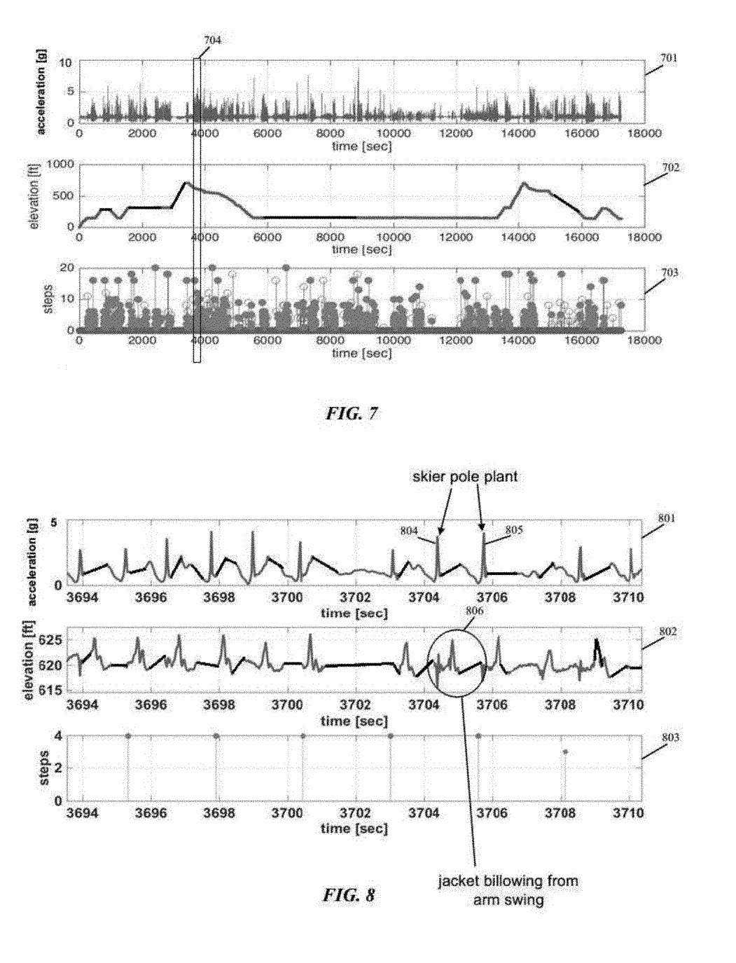

FIG. 7 shows sample motion data that may be generated during a skiing session according to some embodiments of the present disclosure. The top panel shows acceleration data 701 measured by an accelerometer, the middle panel shows elevation data 702 measured by a pressure sensor, and the bottom panel shows step counts 703 measured by a pedometer.

FIG. 8 is a zoomed-in view of the motion data shown in FIG. 7. In particular, acceleration data 801, elevation data 802, and step counts 803 shown in FIG. 8 may correspond to a subset of the acceleration data 701, elevation data 702, and step counts 703 of FIG. 7 indicated by window 704. It can be seen the acceleration data 801 includes a series of peaks 804 and 805, corresponding to points in time when the user planted ski poles. The elevation data 802 includes ripples 806 indicating “jacket billowing” when the user planted the ski poles.

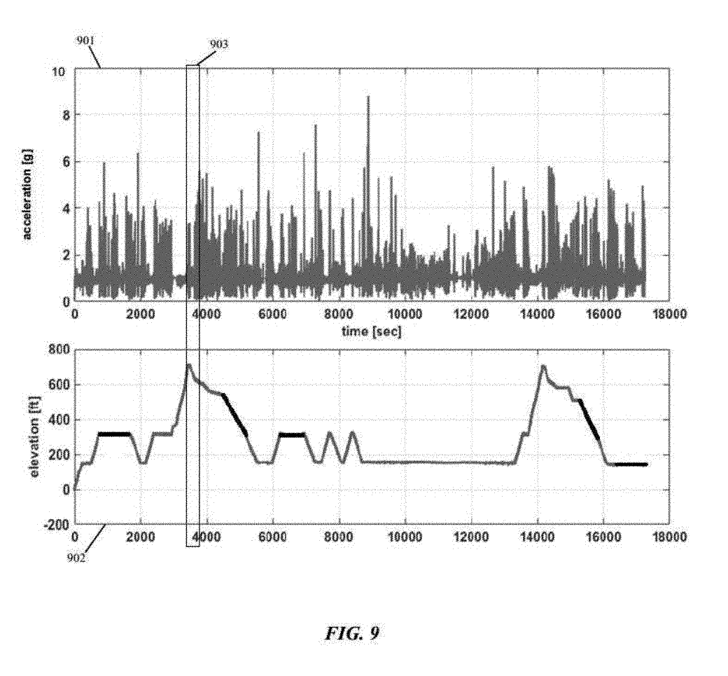

FIG. 9 also shows sample motion data that may be observed during a skiing session according to some embodiments of the present disclosure. The top panel shows acceleration data 901 measured by an accelerometer and the bottom panel shows elevation data 902 measured by a pressure sensor.

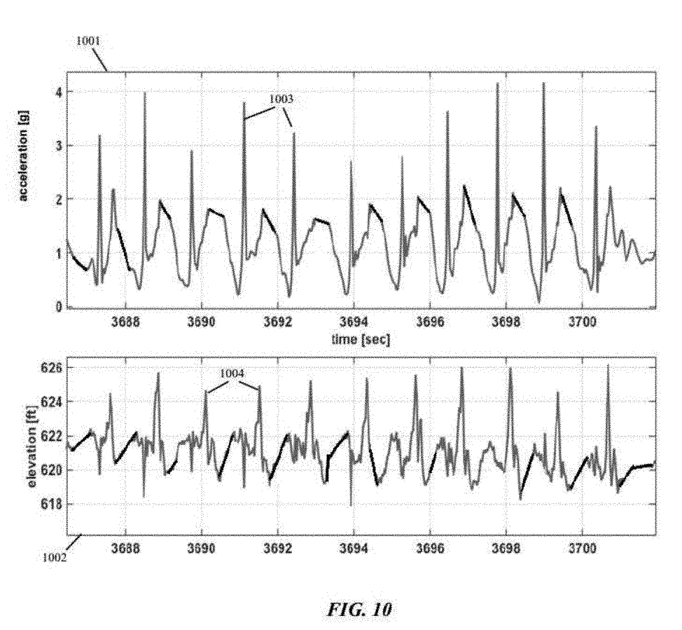

FIG. 10 is a zoomed-in view of the motion data shown in FIG. 9. In particular, acceleration data 1001 and elevation data 1002 shown in FIG. 10 may correspond to a subset of the acceleration data 901 and elevation data 902 of FIG. 9 indicated by window 903. As can be seen, the acceleration data includes peaks 1003 which can be attributed to the user's arm motion when planting ski poles. The elevation data includes ripples 1004 which can be attributed to “jacket billowing” effect.

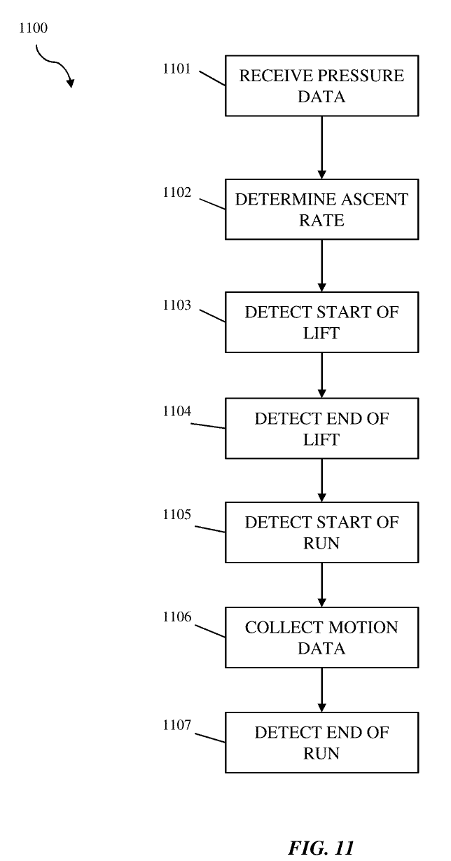

FIG. 11 shows a method 1100 for detecting ski lift/run activity, according to some embodiments of the present disclosure. The method 1100 may be used in combination with method 400 of FIG. 4. For example, method 1100 may be used to determine when to collect/calculate performance information of the user according to step 404 of method 400.

At steps 1101 and 1102, atmospheric pressure data can be received from a pressure sensor and used to determine the user's ascent rate. Ascent rate may be computed as the elevation ascended in a given time interval (Δt), which may be tunable parameter. At step 1103, the user's ascent rate can be compared with a pre-defined ascent rate threshold Rₐ. If the user's ascent rate is greater than the threshold Rₐ, a start of a lift may be detected. In some embodiments, other information factors may be used to detect a start of lift. For example, the wearable/companion device may have access to the location of ski lifts within a ski area and this information may be used to determine when the user approaches a lift base.

In certain embodiments, Rₐ is about 1.31 feet/sec. For example, start of lift detection may require a 2-meter ascent within 5 seconds (i.e., Δt=5), leading to a threshold of Rₐ=1.31 feet/sec. In certain embodiments, elevation updates arrive at a given frequency (e.g., every 2.5 seconds), and the time interval (Δt) is selected to be a multiple of that frequency (e.g., 5 seconds) so as to provide additional robustness to measurement errors and other noise. In some embodiments, lift detection requires at least M consecutive elevation rate computations to exceed the threshold Rₐ. For example, within a 45-second interval, lift detection may require that the ascent rate is above the threshold for at least 25 consecutive seconds. This may increase the true positive rate of lift detection while suppressing false positives.

At step 1104, an end of lift detection is made, meaning that the user is approaching the top of the lift or has exited the lift. Various heuristics may be used to detect the end of lift. In some embodiments, the user's ascent rate may be used to detect end of lift. For example, multiple consecutive ascent rate calculations are below the threshold Rₐ may indicate that the lift is slowing down or has already stopped. In some embodiments, end of lift is detected if at least four out of six 5-second time-windows (20 out of 30 seconds) have ascent rate less than about 1.31 feet/sec. Because ski lifts often slow down or stop for safety reasons, other criteria besides ascent rate may be used to detect end of lift. In particular embodiments, the wearable/companion device may have access to the elevation or change-in-elevation of ski lifts at the ski area and this information can be used, along with measured pressure data, to detect the end of lift.

At step 1105, after an end of lift is detected, a start of a run can be detected. For example, the user's elevation change (e.g., descent rate) can be calculated and compared with a descent rate threshold R_{d}. In certain embodiments, the threshold R_{d} is about −1.31 feet/sec. If the user's descent rate is greater than R_{d}, the start of the run can be detected. In some embodiments, the user's motion data (e.g., step count or arm pose) may be used for run detection. In certain embodiments, a pole plant detector, such as detector 600 of FIG. 6 may be used to detect the start of run.

In many embodiments, components of the wearable device and/or companion device may be powered down while the user is riding a lift or skiing down a run. For example, the main processor 210 may be powered off while the user is riding up a lift to conserve battery charge, while the motion co-processor may remain powered on. As another example, a cellular module within the companion device may be powered down or turned off while the user is skiing down a run.

At step 1106, while the user is on the ski run, motion data of the user can be collected and used to calculate performance information of the skier, such as the number of turns taken, how wide the user's turns are, descent rate, and an overall skiing ability.

At step 1107, an end of the run can be detected. In some embodiments, end of run can be detected when the user's descent rate is below a predetermined threshold and remains so for a certain amount of time (to differentiate mid-run breaks from end of run). In particular embodiments, end of run is detected if the user is relatively inactive or sedentary for a certain amount of time, which can be determined using motion data from the wearable device. In certain embodiments, end of run is detected if the user is inactive for about five (5) minutes. The time period may be selected to be long enough that temporary pauses during a run do not falsely trigger an end of run. In certain embodiments, end of run is detected if the user approaches a lift, which itself can be detected using location data as described above. In one or more embodiments, end of run is detected based on the evaluation drop measured for the user since the start of run. In some embodiments, end of run detection may include using accelerometer data to compute the energy, i.e. intensity of motion, of the user's arm; it is expected that the arm is moving vigorously during a run, whereas during periods of low activity or inactivity, such as standing in line for a lift or taking a snack break, the energy is likely to be low.

In some embodiments, the ski area at which the ski activity is occurring can be determined based on location data received from the GPS module, and a digital elevation model of the ski area can be obtained from an external source. Using the digital elevation model, an expected elevation change associated with a lift can be determined and used to detect an end of lift and/or end of run.

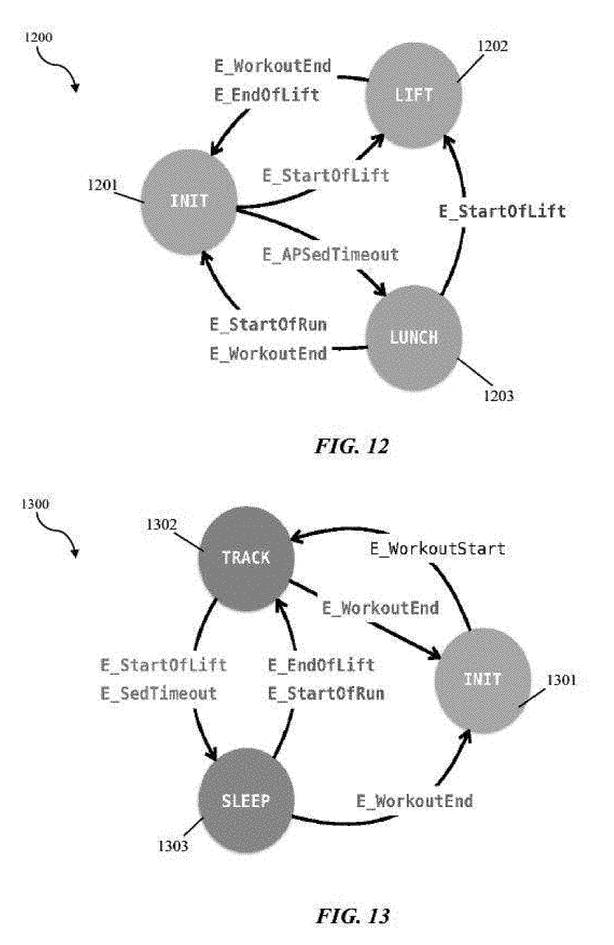

FIGS. 12 and 13 show state machines that may be used within a wearable/companion device to track ski activity, according to some embodiments of the present disclosure. In some embodiments, the state machine 1200 of FIG. 12 corresponds to processing within a motion co-processor, whereas the state machine 1300 of FIG. 13 corresponds to processing within a main processor. State machine 1200 includes an initial state 1201, a lift state 1202, and a break (or “lunch”) state 1203. State machine 1300 includes an initial state 1301, a tracking state 1302, and a sleep state 1303.

The state machines 1200, 1300 begin in respective initial states 1201, 1301. In response to detecting a start of a ski workout (“E_WorkoutStart”), state machine 1300 transitions to tracking state 1302. In some embodiments, the user can provide input to indicate a start and end of a ski workout. Next, in response to detecting a start of lift (“E_StartOfLift”), state machine 1200 transitions to lift state 1202 and state machine 1300 transitions to the sleep state 1303 wherein the main processor may be powered down. Next, if an end of lift is detected (“E_EndOfLift”), state machine 1200 transitions back to initial state 1201 and state machine 1300 transitions to tracking state 1302. State machine 1300 may also transition to tracking state 1302 if a start of run is detected (“E_StartOfRun”). In tracking state 1302, the main processor may be powered on. In many embodiments, the motion co-processor controls when the main processor is powered off and on. For example, once the motion co-processor detects an end of a lift, it can wake up a main processor for tracking. In some embodiments, other components of the wearable/companion device may be powered down in the sleep state 1303, such as the GPS module, cellular module, and/or heart rate sensor.

Next, if an end of run (“E_SedTimeout”) or start of lift (“E_StartOfLift”) is detected, state machine 1300 transitions back to the sleep state 1303. In some embodiments, the end of run may be detected if the user is relatively inactive or sedentary for a pre-determined amount of time (e.g., about five minutes).

State machine 1200 (e.g., the motion co-processor) may transition from the initial state 1201 to the lunch state 1203 if the user is relatively inactive or sedentary (“E_APSedTimeout”) for a pre-determined amount of time. In some embodiments, the timeout used by state machine 1200 (“E_APSedTimeout”) is greater than the timeout used by state machine 1300 (“E_SedTimeout”). From the lunch state 1203, state machine 1200 may transition to lift state 1202 if a start of lift is detected (“E_StartOfLIft”) or to the initial state 1201 if a start of run (“E_StartOfRun”) is detected.

If an end of workout is detected (“E_WorkoutEnd”), state machines 1200 and 1300 transition back to their respective initial states 1201 and 1301.

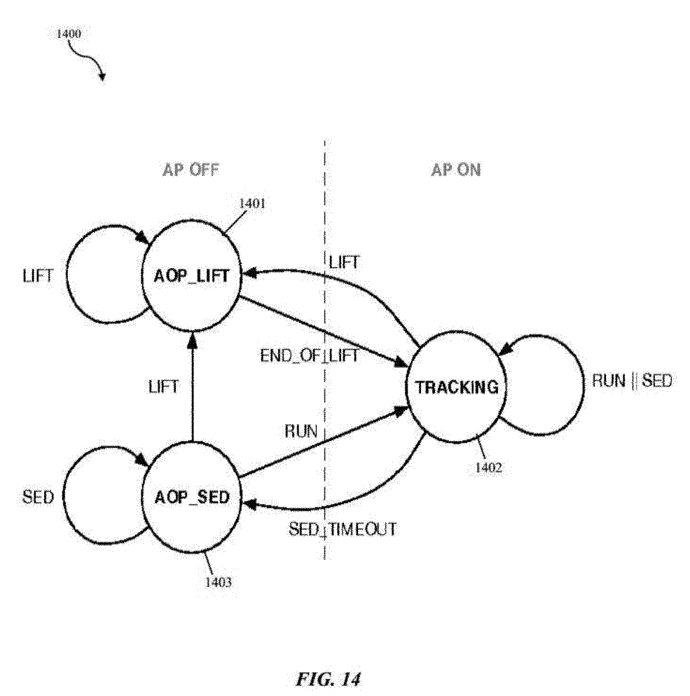

FIG. 14 shows a state machine 1400 according to some embodiments of the present disclosure. The state machine 1400 is partitioned into a first portion (“AP OFF”) corresponding to states when the main processor may be powered down, and a second portion (“AP ON”) corresponding to states when the main processor may be powered on. The first portion comprises a lift state 1401 and a sedentary state 1403, while the second portion comprises a tracking state 1402, as shown.

In some embodiments, a pressure sensor can collect real-time atmospheric pressure data. Based on the collected atmospheric pressure data, the motion co-processor (or “AOP”) can determine an elevation and an elevation rate. In some embodiments, if the motion co-processor detects an elevation rate greater than an ascent rate threshold Rₐ for a period of time, a start of lift is detected (“LIFT”) and the state machine 1400 transitions to lift state 1401. In the lift state 1401, the main processor (and possibly other device components) may be powered down.

In some embodiments, if the motion co-processor detects an elevation rate less than the ascent rate threshold Rₐ for a period of time, it can detect an end of a lift (“END_OF_LIFT”). In response, flow chart 1400 may transition to the tracking state 1402. In some embodiments, the motion co-processor wakes up the main processor. In tracking state 1402, the GPS module and the heart rate sensor can be turned on to monitor the user's activity. The GPS module can collect real-time location data and the main processor can determine the user's skiing route. The user's heart rate can be recorded and used for energy expenditure calculation. In some embodiments, other motion information can also be obtained, such as speed, number of turns, turning rate, descent rate, etc.

The state machine 1400 remains in the tracking state 1402 if a start of run is detected (“RUN”) or if the user is sedentary (“SED”) for less than a threshold amount of time. When there is no significant elevation change or other user motion for a relatively short period of time, it may be assumed that the user is taking a short break, and thus the main processor should continue to track the user's movement. However, if the user is inactive/sedentary for a relatively long period of time (“SED_TIMEOUT”), the it may be assumed that the user has stopped skiing and state machine transitions to the sedentary state 1403 where the main processor can be powered down.

In sedentary state 1403, the motion co-processor can keep monitoring the elevation. If there is an elevation change which can indicate a start of a lift, the motion co-processor can switch to lift state 1401. If there is an elevation change which can indicate a start of a run, the motion co-processor can wake up the main processor to tracking state 1402.

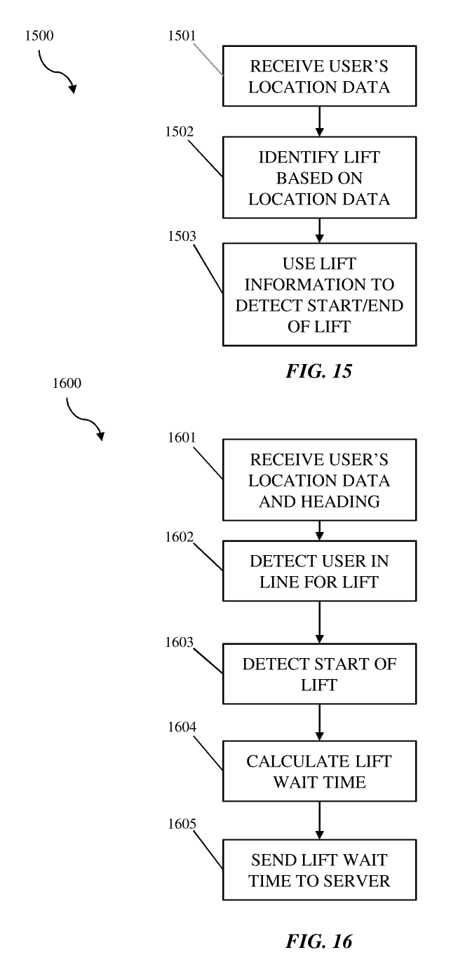

FIG. 15 shows a method 1500 for detecting a start/end of lift based on location data, according to some embodiments of the present disclosure. At step 1501, the user's location may be obtained from a GPS module within the wearable/companion device. In some embodiments, a map or digital elevation model of the ski area can be received, such as from the device's memory or the Internet. In some embodiments, lift location information including start/end location can be determined from the received local maps. At step 1502, the user's location data can be compared with the known lift locations. If the user's location matches that of a lift, the lift which the user is likely to take can be identified. At step 1503, a start and/or an end of the lift can be detected. In some embodiments, other information of the user (e.g., motion data) can be used for lift detection. For example, in some embodiments, a determination as to whether the user sits down can be made based on received motion data, and a start of a lift can be detected based on the determination. Similarly, a determination as to whether the user stands up can be made based on received motion data, and an end of a lift can be detected.

In some embodiments, the user's arm pose angle can be determined based on the user's motion data. For example, a lift can stop half-way through due to certain reasons, the elevation change rate can be nearly zero which is similar to the scenario when the user gets off the lift and starts on a run. However, the user arm pose angle may be substantially unchanged from the time when the user gets on the lift. So it can be determined that the user is still on the lift, and the main processor and/or other modules can remain asleep to conserve battery charge. Thus, a “false wake up” can be avoided.

Once the start of the lift is detected, the main processor and/or other modules can be powered down accordingly to conserve battery charge. At the end of the lift, the main processor and/or other modules can be powered up for tracking.

In some embodiments, the expected lift duration may be determined, such as being retrieved from a local or external data source. Thus the main processor and/or other modules can be powered down to conserve battery charge for a pre-set period of time after the user gest on a lift. When the pre-set period of time is up, the main processor and/or other modules can be powered up for tracking.

In some embodiments, lift information (e.g., location, traveling time, etc.) may be unavailable. The main processor and/or other modules can be powered on during the first time or the first several times the user takes the lift, so the lift information (e.g., location, traveling time, etc.) can be tracked and stored. Based on this information, the main processor and/or other modules can be powered down accordingly during the following lifts to conserve battery charge.

When no nearby cellular tower is available, a cellular module of a mobile device can keep searching for cellular tower with high transmission power, which can drain the battery very quickly. In some embodiments, a map of available cellular access points at the ski area or close to the ski area can be received from an external source. Based on the user's location data, it can then be determined if there is any available cellular access point nearby. If there is no available cellular access point, the cellular module can be turned off to conserve battery charge. In some embodiments, a map of available Wi-Fi access points can be received from an external source. If there is no available Wi-Fi access point nearby, the Wi-Fi module can be turned off to conserve battery charge.

Sometimes, GPS signals can be affected by weather or other factors, leading to location inaccuracy or drift. In some embodiments, one or more turns made by the user can be detected and then be used to enhance the location accuracy, or “smooth out” the GPS data.

FIG. 16 shows a method 1600 for crowdsourcing lift information, according to some embodiments of the disclosure. At step 1601, the user's location data can be received from the GPS module, and the user's heading can be received from the motion sensing module. At step 1602, a lift that the user is likely to take can be identified based on the user's location, the known location of the lift, and the user's heading. At step 1603, a start of the lift can be detected. In some embodiments, the elevation-based detection method can be used. In some embodiments, the user's motion data can be used. At step 1604, a wait time of the lift can be calculated based on the time the user spends in line. At step 1605, the lift wait time can be sent to a server.

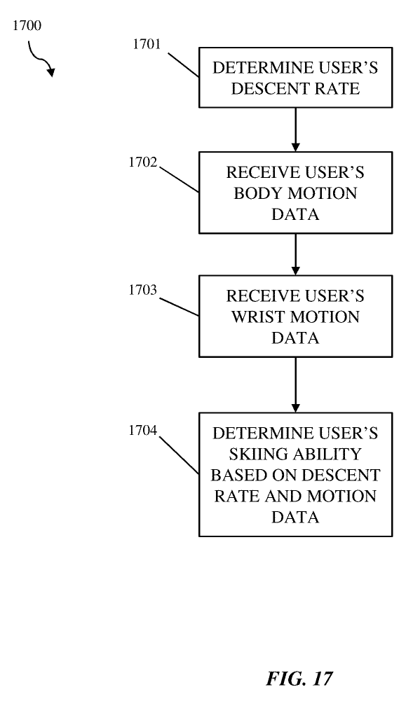

FIG. 17 shows a method 1700 for determining the user's skiing ability, according to some embodiments of the present disclosure. At step 1701, the user's descent rate can be determined based on the user's elevation change. At step 1702, the motion data of the user's body movement can be received. At step 1703, the motion data of the user's wrist movement can be received. In some embodiments, the user can put companion device 300 close his or her torso (e.g., in a pocket, etc.) while strapping wearable device 100 on his or her wrist. Thus companion device 300 can use its motion sensor to track the user's body motion, and wearable device 100 can track the user's wrist motion. At step 1704, the user's skiing ability can be determined based on the user's descent rate and/or the user's motion data.

In some embodiments, the user's skiing ability can be determined by using the user's descent rate. In some embodiments, the user's skiing ability can be determined by both the user's descent rate and location data. For example, an advanced skier's and a beginner may share a start point and an end point during a skiing session. However, the advanced skier's descent rate can be greater than that of the beginner. Thus, by monitoring both the descent rate and the location, the user's skiing ability can be determined.

In some embodiments, the user's skiing ability can be determined by comparing the user's body motion with wrist motion. For example, a strong correlation between a skier's body motion and wrist motion may indicate he or she is an advanced skier, and a weak correlation between a skier's body motion and wrist motion may be a signature of a beginner.

In some embodiments, the user's skiing ability can be determined by the user's turn movement during a skiing session. Performance information such as number of turns, rate of turns, etc., can be obtained based on the user's motion data and used to determine the user's skiing ability. For example, an advanced skier can be more likely to take more turns on a run. Additionally, an advanced skier can be more likely to take tight turns. Thus the number of turns and turn rates can be correlated to a skier's ability.

In some embodiments, a fall of the user can be detected based on the collected motion data. In some embodiments, it can also determine the user's skiing ability based on the detected fall. For example, an advanced skier is less likely to fall, while a beginner is more likely to fall. Sometimes, falls can be life-threatening to a skier and immediate medical care is needed. In some embodiments, once a fall is detected, wearable device 100 or companion device 300 can inform a ski patrol or a first responder.

In some embodiments, an energy expenditure of the user can be calculated based on the determined skiing ability and the calculated energy expenditure can be output to the user.

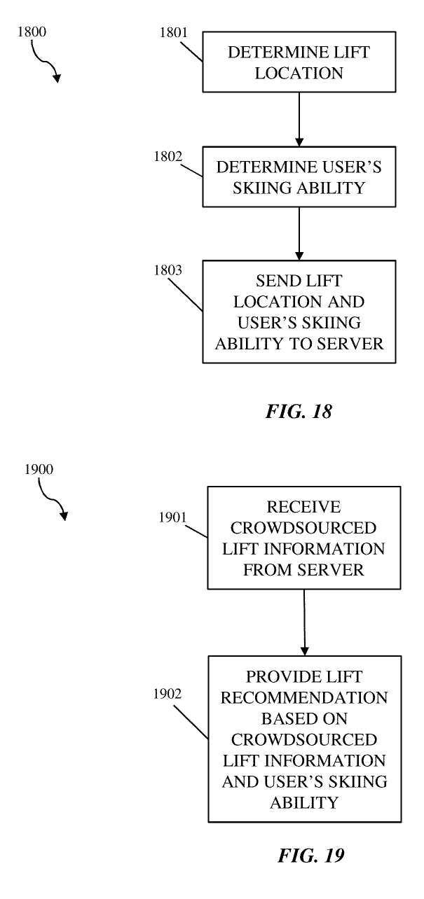

FIG. 18 shows a method 1800 for crowdsourcing lift and skiing ability information, according to some embodiments of the present disclosure. At step 1801, location data of a lift can be determined. In some embodiments, the user's location data can be used for lift detection. In some embodiments, the lift location can be received from an external source (e.g., the Internet, the ski area, etc.) At step 1802, the skiing ability of the user who takes the lift can be determined based on performance information of the user (e.g., descent rate, motion data, etc.) At step 1803, the lift location and the user's ability can be sent to a server. Based on data from a group of users, the server can determine a correlation between a specific lift and the skill levels of users who frequently take this lift. Thus, a difficulty level of the lift can be determined.

FIG. 19 shows a method 1900 for providing a lift recommendation to the user, according to some embodiments of the present disclosure. In some embodiments, the user's skiing ability can be determined first based on the user's motion data in the beginning of a skiing session. In some embodiments, user input can be received regarding his or her skiing ability. At step 1901, crowdsourced lift information can be received (e.g., lift difficulty, lift wait time, etc.) At step 1902, the lift recommendation can be provided to the user based on the received lift information and the user's skiing ability.

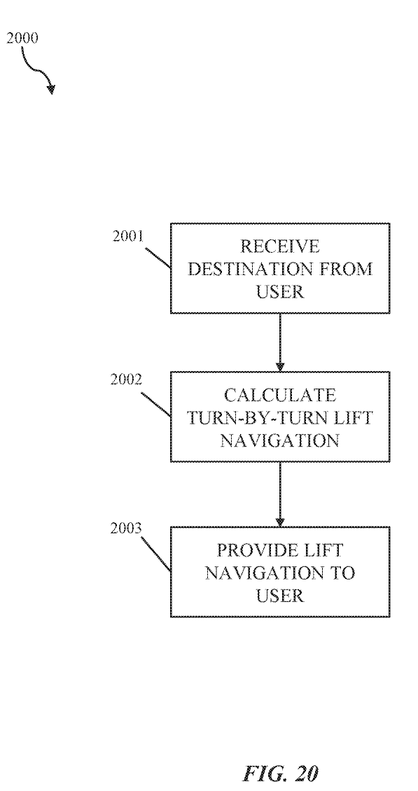

FIG. 20 shows a method 2000 for providing turn-by-turn navigation to the user, according to some embodiments of the present disclosure. At step 2001, the user's input of his or her destination can be received. At step 2002, the turn-by-turn navigation can be calculated based on the user's current location and the user's destination. In some embodiments, other lift information can be used for the navigation calculation (e.g., lift difficulty, lift wait time, etc.) At step 2003, the turn-by-turn navigation can be provided to the user.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of at least one particular implementation in at least one particular environment for at least one particular purpose, those of ordinary skill in the art will recognize that its usefulness is not limited thereto and that the present disclosure may be beneficially implemented in any number of environments for any number of purposes.

Methods described herein may represent processing that occurs within a wearable device (e.g., device 100 of FIG. 1) and/or within a companion device (e.g., device 300 of FIG. 3). In many embodiments, processing may be performed by a processor circuit within the wearable/companion device. In some embodiments, processing may be performed using computer software instructions executed upon a computer processor. In particular embodiments, the computer software instructions may be provided on a non-transitory computer-readable medium. In certain embodiments, processing may be performed using a digital signal processor (DSP) circuit or an application specific integrated circuit (ASIC).

What is claimed is:

1. A method for improving an accuracy of a wearable device while calculating performance information of a skier, the method comprising:
receiving motion data of a user from a motion sensing module of the wearable device;
measuring, by a heart rate sensing module of the wearable device, a heart rate of the user, wherein the heart rate sensing module comprises a photoplethysmogram (PPG) sensor and the PPG sensor is configured to be worn adjacent to the user's skin;
detecting, by one or more processor circuits of the wearable device, a start of the ski activity by the user, wherein detecting the start of the ski activity comprises determining the user is gripping ski poles based on the motion data and the heart rate of the user;
calculating, by the one or more processor circuits, the user's performance information during the ski activity; and
outputting, by the one or more processor circuits, the calculated performance information.

2. The method of claim 1, comprising:
receiving atmospheric pressure data from a pressure sensor of the wearable device;
determining, by the one or more processor circuits, an ascent rate based on the received atmospheric pressure data;
comparing, by the one or more processor circuits, the ascent rate with an ascent rate threshold; and
detecting, by the one or more processor circuits, a start of a lift based upon comparing the ascent rate with the ascent rate threshold.

3. The method of claim 2, comprising:
in response to the detected start of the lift, decreasing a power level of at least one of the one or more processor circuits.

4. The method of claim 2, comprising:
determining, by the one or more processor circuits, an expected elevation change associated with the lift;
determining, by the one or more processor circuits, an elevation change of the user based on the received atmospheric pressure data;
comparing, by the one or more processor circuits, the elevation change of the user with the expected elevation change associated with the lift; and
detecting, by the one or more processor circuits, an end of the lift based upon comparing the elevation change of the user with the expected elevation change.

5. The method of claim 4, wherein determining the expected elevation change associated with the lift comprising:
receiving location data from a GPS module of the wearable device;
determining, by the one or more processor circuits, a ski area at which the ski activity is occurring based on the location data;
receiving, by the one or more processor circuits, a digital elevation model of the ski area; and
determining, by the one or more processor circuits, the expected elevation change associated with the lift based on the received digital elevation model.

6. The method of claim 4, comprising:
in response to the detected end of the lift, increasing a power level of at least one of the one or more processor circuits.

7. The method of claim 1, comprising:
receiving atmospheric pressure data from a pressure sensor of the wearable device;
determining, by the one or more processor circuits, an descent rate based on the received atmospheric pressure data;
comparing, by the one or more processor circuits, the descent rate with an descent rate threshold; and
detecting, by the one or more processor circuits, a start of a run based upon comparing the descent rate with the descent rate threshold.

8. The method of claim 7, comprising:
in response to the detected start of the run, turning off, by the one or more processor circuits, at least one of a cellular module or a Wi-Fi module of the wearable device.

9. The method of claim 1, wherein the user's performance information comprises at least one of a number of runs, a length of runs, an overall distance skied, an elevation drop, or a speed.

10. The method of claim 1, comprising:
detecting, by the one or more processor circuits, the user is in a seated position based on the received motion data;
in response to the detection that the user is in the seated position, determining, by the one or more processor circuits, a start of a lift;
detecting, by the one or more processor circuits, the user stands up; and
in response to the detection that the user stands up, determining, by the one or more processor circuits, an end of the lift.

11. The method of claim 1, comprising:
receive location data of the user from a GPS module of the wearable device;
receiving a heading of the user from the motion sensing module; and
determining, by the one or more processor circuits, a lift which the user is in line for based on the received location data and heading.

12. The method of claim 1, comprising:
detecting, by the one or more processor circuits, the user is sedentary based on the motion data; and
in response to the detection that the user is sedentary, determining by the one or more processor circuits, an end of a run.

13. The method of claim 1, comprising:
detecting, by the one or more processor circuits, the user has experienced a fall based on the motion data; and
in response to a detected user's fall, notifying, by the one or more processor circuits, a ski patrol.

14. The method of claim 1, comprising:
receiving, by the one or more processor circuits, a location of a cellular access point;
receiving, by the one or more processor circuits, location data from a GPS module of the wearable device; and
disabling, by the one or more processor circuits, a cellular module based upon comparing the received location data with the location of the cellular access point.

15. The method of claim 1, comprising:
determining, by the one or more processor circuits, a skiing ability for the user based on the motion data; and
outputting, by the one or more processor circuits, a lift recommendation based on the user's skiing ability.

16. The method of claim 15, comprising:
calculating, by the one or more processor circuits, an energy expenditure of the user based on the determined skiing ability of the user; and
outputting, by the one or more processor circuits, the energy expenditure of the user.

17. The method of claim 15, wherein determining the skiing ability of the user comprises:
detecting, by the one or more processor circuits, a number of falls the user has experienced based on the motion data; and
determine the skiing ability of the user based on the detected number of falls.

18. The method of claim 15, wherein determining the skiing ability of the user comprises:
determining, by the one or more processor circuits, a turn rate based on the motion data; and
determining, by the one or more processor circuits, the skiing ability of the user based on turn rate.

19. The method of claim 15, wherein determining the skiing ability of the user comprises:
receiving, atmospheric pressure data from a pressure sensor of the wearable device;
determining, by the one or more processor circuits, a descent rate based on the received atmospheric pressure data; and
determining, by the one or more processor circuits, the skiing ability for the user based on the determined descent rate.

20. The method of claim 15, wherein determining the skiing ability of the user comprises:
receiving from a first motion sensing module of the wearable device, a first set of motion data;
receiving from a second motion sensing module of a companion device, a second set of motion data;
comparing, by the one or more processor circuits, the first set of motion data and the second set of motion data; and
determining, by the one or more processor circuits, the skiing ability for the user based upon comparing the first set of motion data and the second set of motion data.

21. The method of claim 15, comprising:
receiving, by the one or more processor circuits, crowdsourced lift information from an external source; and
outputting, by the one or more processor circuits, a lift recommendation based on the user's skiing ability and the crowdsourced lift information.

22. The method of claim 1, comprising:
receiving location data from a GPS module of the wearable device;
determining, by the one or more processor circuits, a proximity to a lift based on the received location data;
receiving atmospheric pressure data from a pressure sensor of the wearable device;
detecting, by the one or more processor circuits, a start of the lift based on the atmospheric pressure data;
determining, by the one or more processor circuits, a lift waiting time based on the determined proximity to the lift and the detected start of the lift; and
sending, by the one or more processor circuits, the determined lift waiting time to a server.

23. The method of claim 1, comprising:
receiving, by the one or more processor circuits, crowdsourced lift information from an external source;
receiving, by the one or more processor circuits, an input from the user regarding a destination; and
outputting, by the one or more processor circuits, a turn-by-turn navigation based on the crowdsourced lift information and the received input.

24. A system for improving an accuracy of a wearable device while calculating performance information of a skier, the system comprising:
a motion sensing module configured to collect a user's motion data;
a heart rate sensing module configured to measure a heart rate of the user, wherein the heart rate sensing module comprises a photoplethysmogram (PPG) sensor and the PPG sensor is configured to be worn adjacent to the user's skin;
a processor circuit in communication with the motion sensing module and the heart rate sensing module and configured to execute instructions causing the processor circuits to:
detect a start of the ski activity by the user, wherein detecting the start of the ski activity comprises determining the user is gripping ski poles based on the motion data and the heart rate of the user;
calculate performance information about the user during the ski activity; and
output the calculated performance information.
